(19) United States
(12) Patent Application Publication
(10) Pub. No.: US 2026/0359981 A1
(43) Pub. Date: Nov. 12, 2026

(54) RetinalGPT: A Retinal Clinical Preference Conversational Assistant Powered by Large Vision-Language Models

(71) Applicants: Arizona Board of Regents on behalf of Arizona State University, Scottsdale, AZ (US); Mayo Foundation for Medical Education and Research, Rochester, MN (US)

(72) Inventors: Yalin WANG, Tempe, AZ (US); Xin LI, Dalian (CN); Wenhui ZHU, Tempe, AZ (US); Oana DUMITRASCU, Scottsdale, AZ (US)

(21) Appl. No.: 19/669,875

(22) Filed: May 6, 2026

Related U.S. Application Data
(60) Provisional application No. 63/801,001, filed on May 6, 2025.

(51) Int. Cl. G06T 7/00; G06V 10/774; G06V 10/94; G16H 30/20

(52) U.S. Cl. CPC G06T 7/0012; G06V 10/774; G06V 10/945; G16H 30/20; G06T 2207/20036; G06T 2207/20081; G06T 2207/30041; G06T 2207/30096; G06T 2207/30101; G06T 2207/30168; G06V 2201/03

(57) ABSTRACT

Retinal clinical preference large-vision language model conversational assistant that receives a plurality of retinal images, and extracts clinical feature descriptions, including disease labels, lesion bounding boxes, and fractal features, from the plurality of retinal images. The assistant fractal analyzes each retinal image to extract features including fractal dimension of vessels, vessel density, branch angle, curvature tortuosity, and morphological characteristics relevant to vascular structure. The assistant statistically analyzes the extracted features to examine their relationship with disease labels and to ensure the extracted features are clinically relevant.

### CLAIM OF PRIORITY

This application claims the benefit of U.S. Provisional Patent Application No. 63/801,001, filed May 6, 2025, entitled “RETINAL GPT: A RETINAL CLINICAL PREFERENCE CONVERSATIONAL ASSISTANT POWERED BY LARGE VISION-LANGUAGE MODELS”, the disclosure of which is incorporated by reference herein in its entirety.

### COPYRIGHT NOTICE

A portion of this document contains material which is subject to copyright protection. The copyright owner has no objection to the facsimile reproduction by anyone of the document as it appears in the Patent and Trademark Office records but otherwise reserves all copyright rights whatsoever.

### TECHNICAL FIELD

The disclosed embodiments relate to a generative AI model for multimodal conversational assistant for clinically preferred quantitative analysis of retinal images.

### BACKGROUND

Retinal color fundus photography (CFP) plays a central role in the clinical evaluation of ocular diseases. With the increasing adoption of non-mydriatic CFP systems, retinal imaging has become more accessible for point-of-care screening and longitudinal assessment. Over the past decade, deep learning approaches, particularly convolutional neural networks (CNNs), have achieved strong performance in automated retinal disease detection. However, most CNN-based methods are limited to closed-set classification and provide limited support for clinically interpretable and quantitative analysis, which remains essential for complex ophthalmic assessment.

Recent advances in large language models (LLMs) have driven the development of vision-language models (VLMs) that integrate visual perception with natural language understanding. Several general-purpose and medical VLMs have demonstrated promising capability in multimodal medical interpretation. Despite this progress, existing medical VLMs remain insufficient for specialized ophthalmic applications.

In clinical practice, retinal interpretation often relies on fine-grained analysis of vascular morphology and lesion distribution, including quantitative biomarkers such as vessel density, fractal dimension, tortuosity, and branching patterns. These features are critical for assessing diabetic retinopathy, hypertensive retinopathy, and emerging retinal indicators associated with neurodegenerative conditions. Existing medical VLMs, lacking training on structured analytical instruction data, struggle with this level of clinical preference reasoning. Furthermore, they frequently exhibit an inability to accurately localize abnormalities, including microaneurysms, hemorrhages, and hard exudates, which is vital for interpretable diagnosis. Another pervasive challenge is the catastrophic forgetting of generic-domain knowledge during the fine-tuning process, which limits the model's overall versatility.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 depicts a multi-round interactive discussion between the user and RetinalGPT for retinal image analysis, in which the user can engage with RetinalGPT to iteratively explore disease-related findings, vascular characteristics, and lesion localization based on a given fundus image, according to the disclosed embodiments.

FIG. 2A illustrates a process of obtaining data according to the disclosed embodiments.

FIG. 2B shows the collected data is converted into instruction-following data, categorized into alignment, according to the disclosed embodiments.

FIG. 2C shows collected data converted into tuning data, according to the disclosed embodiments.

FIG. 3 illustrates an overview of the model architecture and training strategy according to the disclosed embodiments.

FIG. 4A shows the disclosed embodiments can predict lesion locations with ground truth annotations.

FIG. 4B presents vascular structure analysis where the disclosed embodiments can estimate fractal dimension, vessel density, and vessel width and compare them against ground truth values.

FIG. 5 compares the disclosed embodiments (Retinal GPT) with LLaVA-Med on a generic medical domain. The underlined sections illustrate highly similar parts of the responses. The disclosed embodiments demonstrate the ability to preserve knowledge in the generic medical domain.

FIG. 6 presents Table 1, which compares the performance of different models on abnormality classification.

FIG. 7 presents Table 2, which provides a quantitative comparison of diagnostic performance and response quality.

FIG. 8 presents Table 3, which provides a quantitative comparison between predicted vascular features and AutoMorph reference measurements.

FIG. 9 presents Table 4, which provides a quantitative evaluation of lesion localization performance on IDRiD and MICCAI datasets under two center-distance thresholds (α=0.5 and α=0.8).

### DETAILED DESCRIPTION

### 1. Introduction

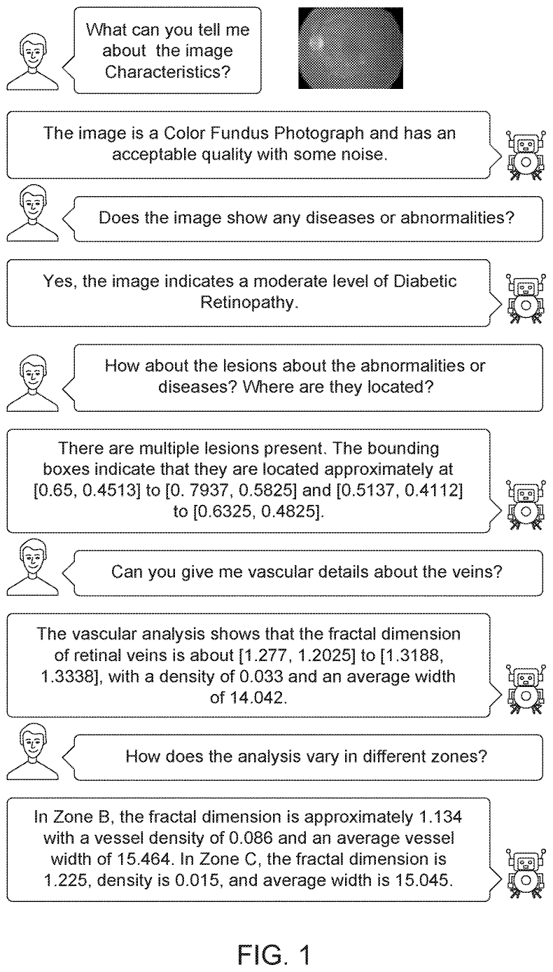

To address the above-described challenges in the prior art, the disclosed embodiments present RetinalGPT, a multimodal assistant designed to support interactive, image-centered retinal analysis. As illustrated in FIG. 1, RetinalGPT enables a clinician-patient-like multi-round interaction, allowing users to progressively explore disease-related findings, lesion localization, and quantitative vascular characteristics from a single fundus image. By integrating large-scale retinal data curation, a dedicated quantitative analysis pipeline, and customized visual instruction tuning, RetinalGPT aligns multimodal perception with expert ophthalmic reasoning, providing a coherent and interpretable paradigm for systematic retinal image analysis.

Quantitative analysis of retinal vasculature, including metrics such as vessel density and fractal dimension, is essential for evaluating diabetic retinopathy (DR) and investigating emerging retinal biomarkers associated with systemic diseases such as Alzheimer's disease (AD). However, existing multimodal medical language models lack the capability to perform fine-grained vascular quantification and image-grounded interpretation from color fundus photographs (CFPs). The disclosed embodiments leverage a large language model as a multimodal reasoning engine for quantitative retinal analysis, enabling precise vascular measurement and clinically meaningful interpretation from CFPs.

The disclosed embodiments provide for RetinalGPT, a multimodal conversational assistant for clinically preferred quantitative analysis of retinal images. The system integrates (1) a large-scale, high-quality CFP dataset, (2) an automated retinal morphology quantification pipeline, and (3) customized visual instruction tuning to align visual representations with expert ophthalmic knowledge. The disclosed embodiments enable interactive, image-centered analysis, supporting disease-related assessment, lesion localization, and quantitative vascular characterization within a unified analytical paradigm.

Comprehensive evaluations across eight benchmark retinal datasets demonstrate that RetinalGPT consistently outperforms state-of-the-art general-domain multimodal language models in ophthalmic and AD-related classification tasks. Beyond classification, RetinalGPT accurately localizes retinal lesions such as hemorrhages and exudates and produces quantitative vascular measurements, including fractal dimension, that are consistent with established clinical analysis standards.

RetinalGPT provides an interpretable and quantitative framework for retinal image analysis, enabling integrated assessment of retinal pathology and vascular structure, highlighting the potential of multimodal conversational systems to support quantitative retinal research and the exploration of retinal biomarkers relevant to systemic diseases.

### 2. Related Work

### 2.1 General Multi-Modal Large Language Models

Recent advances in visual instruction tuning have significantly accelerated the development of general multimodal large language models (MLLMs) by enabling effective alignment between vision encoders and large language models (LLMs). Early representative approaches, such as BLIP-225, introduced architectural mechanisms to bridge visual and linguistic representations, laying the foundation for multimodal reasoning. Subsequent works, including LLaVA and InstructBLIP, demonstrated that high-quality visual instruction data and lightweight projection strategies are sufficient to unlock strong zero-shot and interactive multimodal capabilities in pre-trained LLMs. Collectively, these studies illustrate the rapid evolution of instruction-tuned multimodal models toward versatile, general-purpose visual-language assistants.

### 2.2 Biomedical Visual Language Models

While general-domain MLLMs exhibit impressive capabilities, their direct application to the medical field is often hindered by a domain gap. To address this, a growing body of research has focused on adapting these frameworks specifically for biomedical tasks. LLaVA-Med, for instance, extended the LLaVA architecture by incorporating a curriculum learning strategy and training on a vast corpus of biomedical image-text pairs derived from open-access literature (e.g., PMC-15M). This adaptation proved that instruction tuning is critical for enabling domain-specific multimodal dialogue and achieving strong performance across various biomedical benchmarks. However, despite these advances, current medical VLMs primarily focus on broad biomedical concepts. They often lack the depth required for highly specialized clinical fields like ophthalmology, where interpretation relies not just on general recognition, but on precise, fine-grained analysis of anatomical structures.

### 2.3 Deep Learning and Quantitative Analysis in Retinal Imaging

Prior to the emergence of multimodal language models, convolutional neural networks (CNNs) were the dominant paradigm for retinal image analysis, achieving strong performance in disease detection and segmentation tasks. Beyond disease classification, a growing body of clinical research has highlighted the retinal vasculature as an important indicator of both ocular and systemic health. Quantitative vascular features, including vessel caliber, tortuosity, branching geometry, and fractal dimension, have been associated with diabetic retinopathy, glaucoma, hypertension, and neurodegenerative disorders such as Alzheimer's disease. Extracting these biomarkers remains challenging, as accurate estimation requires high-fidelity vessel segmentation and precise morphological measurement, which are labor-intensive and prone to observer variability when performed manually. To address these challenges, automated pipelines such as AutoMorph and RBAD have been proposed to standardize retinal vascular morphology quantification and branching analysis. While these methods provide rigorous quantitative measurements, they operate independently of higher-level clinical interpretation. In contrast, the disclosed embodiments bridge quantitative vascular analysis with interactive multimodal reasoning, enabling clinically interpretable exploration of retinal images within a unified analytical framework.

### 3. Methods

### 3.1 Overview of the Proposed Framework

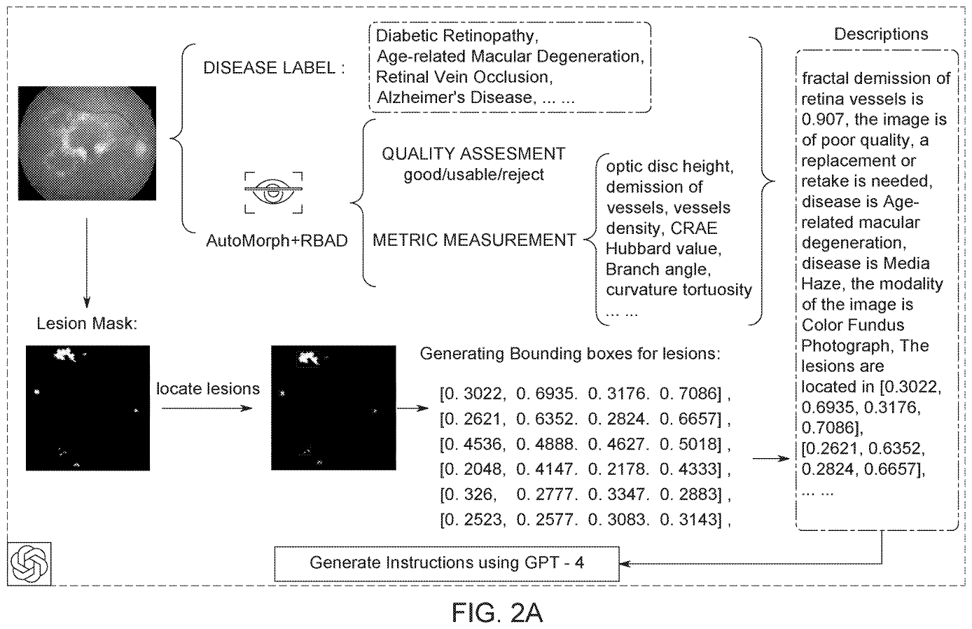

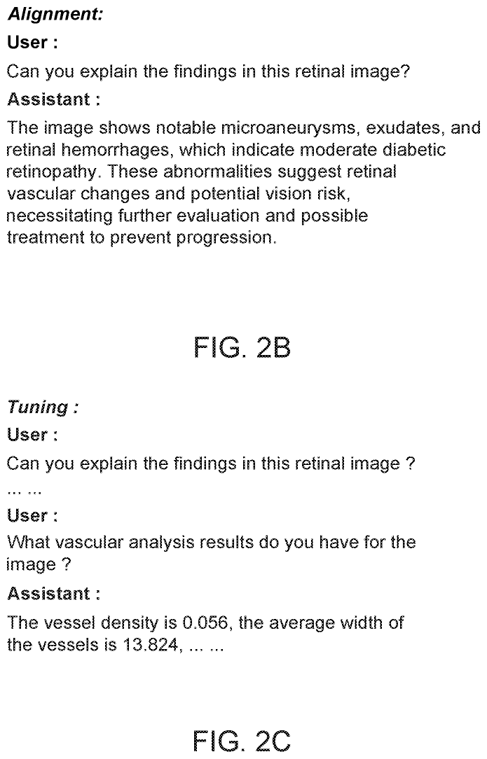

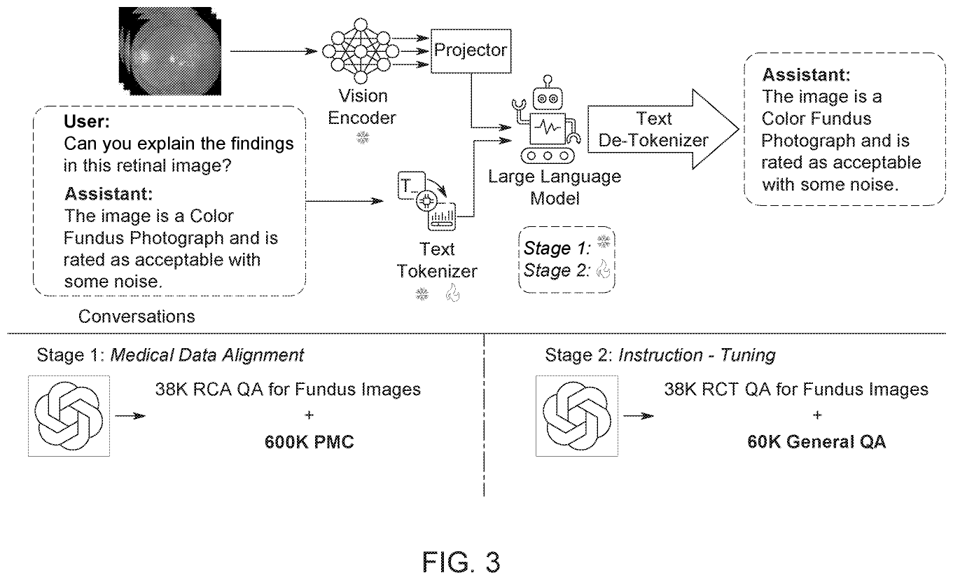

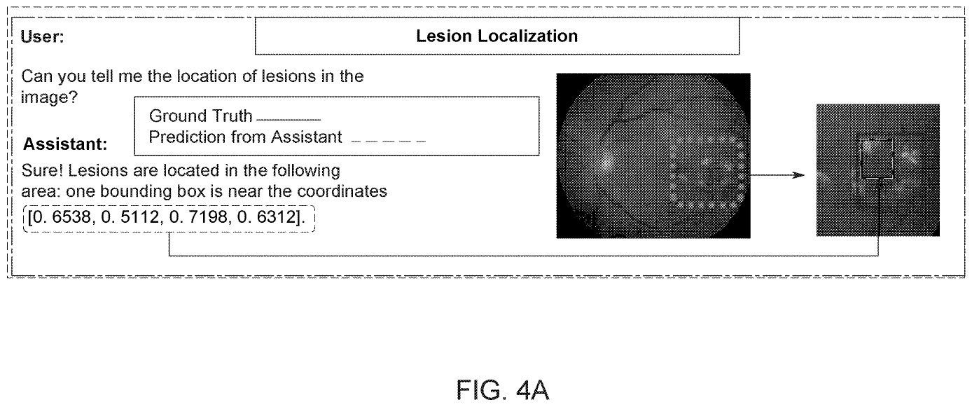

To bridge the gap between general-domain MLLMs and the specialized requirements of ophthalmology, the disclosed embodiments provide a comprehensive framework comprising a clinically grounded data pipeline (FIGS. 2A-2C) and a two-stage training strategy (FIG. 3). The disclosed embodiments focus on embedding quantitative vascular morphology and lesion-aware knowledge into the model's reasoning process.

### 3.2 Clinical Data Construction Pipeline

The core aspect of the disclosed embodiments lies in the construction of a high-quality, instruction-following dataset that incorporates expert-level clinical preferences. The data construction pipeline consists of two phases: Clinical Feature Extraction and Visual Instruction Generation.

### 3.2.1 Clinical Feature Extraction and Quantitative Analysis

Unlike generic image captioning, clinical interpretation requires precise quantification. A large-scale collection of retinal images was compiled from both open-access and private sources. For each image I, a structured clinical feature was extracted, shown in FIG. 2A, as the set: S_{clinical}={X_{d}, X_{q}, X_{f}, Xₗ, Xₘ}
Disease Labels (X_{d}): Ground-truth diagnostic labels derived from medical annotations.
Quantitative Vascular Features (X_{f}): To capture morphological subtleties, automated pipelines were used, specifically AutoMorph and RBAD, to perform fractal analysis. Quantitative indices were extracted, including vessel fractal dimension, vessel density, branching angle, and curvature tortuosity. To ensure clinical relevance, statistical analyses were conducted and ophthalmologists consulted, filtering out redundant metrics and retaining approximately 40 significant features that strongly correlate with disease pathology.
Image Quality Assessment (X_{q}): Images were categorized into “good,” “usable,” or “reject” to ensure data reliability.
Lesion Localization (Xₗ): For datasets with pixel-level annotations, bounding boxes B=(xₘᵢₙ, yₘᵢₙ, xₘₐₓ, yₘₐₓ) were generated to represent lesion locations.
Modality Information (Xₘ): Metadata specifying the imaging modality (Color Fundus Photography).

After filtering out low-quality images unsuitable for analysis, a refined set of approximately 38,000 images with their corresponding structured descriptions was curated.

### 3.2.2 Visual Instruction Data Generation

GPT-4 was leveraged to convert the structured clinical features into natural language instruction-following data, shown in FIG. 2B.
Retinal Concepts Alignment (D_{align}): To align visual features with clinical concepts, single-turn QA pairs were generated. Using the disease label X_{d} and modality Xₘ, GPT-4 was prompted to create concise inquiries (e.g., “What does this image show?”) and corresponding answers. This resulted in the Retinal Concepts Alignment (38K RCA) dataset.
Clinical Preference Instruction-Tuning Data (Dₜᵤₙₑ): To enable complex reasoning, multi-turn dialogues were generated using the full feature set S_{clinical}. Task-specific prompts were designed for scenarios such as Diabetic Retinopathy grading, multi-disease classification, and lesion localization. Crucially, an “expert” response style was simulated where GPT-4 reasoned based on the quantitative features X_{f} and lesion coordinates Xₗ. This process yielded the Retinal Concepts Tuning (38K RCT) dataset.

### 3.2.3 Model Architecture

A state-of-the-art Multimodal Large Language Model architecture (FIG. 3) was adopted. The system consists of three primary components:
Vision Encoder: A pre-trained CLIP model (ViT-L/14) serves as the visual feature extractor, encoding the input retinal image Xᵥ into visual embeddings Zᵥ.
Language Model: A LLaMA was used as the language backbone to handle textual inputs and generate responses.
Projector: A linear projection layer connects the vision encoder and the LLM, mapping visual embeddings Zᵥ into the word embedding space of the language model.

### 3.3 Two-Stage Training Strategy

To mitigate the risk of catastrophic forgetting—where the model loses generic medical knowledge while learning retinal specifics—a “Mixup” training strategy was implemented across two stages.
Stage 1: Feature Alignment. The goal of this stage is to align retinal visual features with the LLM's embedding space while preserving broader biomedical understanding. A mixup dataset was constructed that combines a 38K RCA dataset with 600K image-text pairs from PMC-15M. During training, the Vision Encoder and the LLM were frozen, updating only the parameters of the Projector layer. This ensures that the projection layer learns to map both general biomedical and specific retinal features to the appropriate textual tokens.
Stage 2: Clinical Preference Instruction Tuning. In the second stage, the model was fine tuned to follow complex clinical instructions. A mixup dataset was used comprising a 38K RCT dataset and 60K Generic QA pairs (covering modalities like CXR, CT, and MRI) from LLaVA-Med. During this phase, the Vision Encoder remained frozen, while both the Projector and the LLM weights were updated. This strategy allows the model to internalize the nuanced, quantitative reasoning patterns required for retinal analysis without overfitting to a single domain, thereby maintaining its versatility as a general medical assistant.

### 4. Experiments and Results

### 4.1 Datasets

To ensure robust model training and rigorous evaluation across diverse imaging modalities, demographics, and clinical pathologies, a unified benchmark was compiled from eight mainstream retinal datasets. This collection encompasses a broad spectrum of clinical tasks, ranging from fine-grained disease staging to multi-label recognition and systemic biomarker identification. The characteristics of the included datasets are summarized as follows:
APTOS 2019: The APTOS 2019 (Asia Pacific Tele-Ophthalmology Society) dataset evaluates performance on real-world clinical data, comprising 3,662 high-resolution fundus images from Aravind Eye Hospital, India. To reflect diverse clinical challenges, images were captured across multiple rural clinics using various camera models under heterogeneous conditions. Medical professionals graded each image via the International Clinical Diabetic Retinopathy (ICDR) scale, categorizing severity into five stages: No DR (Grade 0), Mild (Grade 1), Moderate (Grade 2), Severe (Grade 3), and Proliferative DR (Grade 4).
EyePACS/EyeQ: The EyeQ dataset is a large-scale subset of 28,792 photographs derived from the EyePACS repository. Distinct from the original collection's focus on disease grading, EyeQ is curated for Image Quality Assessment (IQA) via a two-tier expert annotation system. First, images are classified into three quality tiers (Good, Usable, and Reject) based on illumination, clarity, and the visibility of key landmarks such as the optic disc and macula. Second, images are staged for Diabetic Retinopathy (DR) using the ICDR scale across five levels: No DR (0), Mild (1), Moderate (2), Severe (3), and Proliferative DR (4). The dataset encapsulates diverse real-world artifacts, including non-standardized lighting, blurring, and varying fields of view characteristic of field-based screening.
Messidor: The Messidor (Methods to Evaluate Segmentation and Indexing Techniques in the field of Retinal Ophthalmology) database comprises 1,200 CFPs acquired by three French ophthalmologic departments using a Topcon TRC NW6 non-mydriatic camera with a 45° field of view. To ensure diversity, the dataset includes varying resolutions (1440×960 to 2304×1536 pixels) captured both with (n=800, 0.5% Tropicamide) and without (n=400) pharmacological pupil dilation. Expert ophthalmologists established ground truth labels using specialized clinical scales: Diabetic Retinopathy (DR) is staged into four levels (0-3) by quantifying microaneurysms, hemorrhages, and neovascularization, while Macular Edema (ME) risk is stratified into three levels (0-2) based on the minimum distance between hard exudates and the macula.
IDRiD: The Indian Diabetic Retinopathy Image Dataset (IDRiD) provides a benchmark for the clinical grading of DR and DME, consisting of high-resolution CFPs (4288×2848 pixels) acquired at a clinic in Nanded, India, using a Kowa VX-10α camera with a 50° FOV. A distinguishing feature is its multi-task annotation framework: the lesion segmentation task provides pixel-level ground truth for four primary lesions—microaneurysms, hemorrhages, hard exudates, and soft exudates—while the clinical grading task stages DR severity via the ICDR scale (0-4) and DME risk (Grades 0-2) based on the distance between hard exudates and the macula. All images were expert-verified to ensure accuracy in both pathological localization and diagnostic classification.
OIA-ODIR: OIA-ODIR (Ocular Disease Intelligent Recognition) is a multi-institutional retinal image dataset designed to simulate real-world clinical diagnostic scenarios. It comprises clinical data from 3,500 patients, providing a total of 7,000 color fundus photographs organized as bilateral pairs (left and right eyes for each patient). The dataset was collected from multiple medical centers using various fundus cameras, including models from Canon, Zeiss, and Topcon, resulting in diverse image resolutions and quality variations. Each patient record was annotated by trained ophthalmologists into eight categories: Normal (N), Diabetes (D), Glaucoma (G), Cataract (C), Age-related macular degeneration (A), Hypertension (H), Pathological Myopia (M), and Other diseases/abnormalities (O). This dataset is characterized by its patient-level multi-label classification format, reflecting the complexity of co-occurring ocular conditions in clinical practice.
RFMiD: The Retinal Fundus Multi-Disease Image Dataset (RFMiD) consists of 3,207 CFPs encompassing 28 distinct retinal pathological conditions. To ensure clinical diversity, images were captured using three camera models—Topcon TRC-NW8F, Topcon TRC-50DX, and Kowa VX-10—with field-of-view angles of 30°, 45°, and 50°. Beyond common conditions like DR, AMD, and glaucoma, the dataset includes expert-verified rare pathologies such as retinal vein occlusion and retinal detachment. This high degree of disease variety serves as a benchmark for evaluating diagnostic generalization across a broad clinical spectrum.
MICCAI Challenge Datasets: The MMAC (Multi-modal Multi-task Age-related Macular Degeneration Challenge) dataset from MICCAI 2023 provides a multi-modal repository of CFPs and OCT images for studying pathological myopia (PM) and AMD. For myopia classification, images are staged from C0 to C4 according to the Meta-PM grading standard. For AMD, the dataset includes task-specific annotations for classification and axial length prediction. Its multi-institutional nature and multi-task labeling provide a rigorous framework for evaluating diagnostic performance in complex high-myopia-related retinal pathologies.
UK Biobank: The UK Biobank was utilized for the analysis of Alzheimer's Disease (AD) and Vascular Dementia (VD). The cohort includes 1,136 CFPs from AD patients and 342 CFPs from VD patients, with clinical diagnoses verified via ICD-10 codes from hospital admission and death records.

To ensure data consistency and eliminate domain bias, all color fundus photographs (CFPs) underwent a standardized preprocessing pipeline, localized via Hough circle transform, followed by mask-area cropping and resolution unification. The disclosed embodiments further implemented a rigorous quality control protocol using AutoMorph to exclude “Reject” tier images that preclude accurate vascular quantification. Following this filtration, 41,815 high-quality images from eight datasets were retained: APTOS 2019 (n=3,661), EyePACS/EyeQ (n=23,223), Messidor (n=1,200), IDRiD (n=597), OIA-ODIR (n=6,977), RFMiD (n=3,199), MMAC (n=1,571), and UK Biobank (n=1,387).

### 4.2 Data Splitting

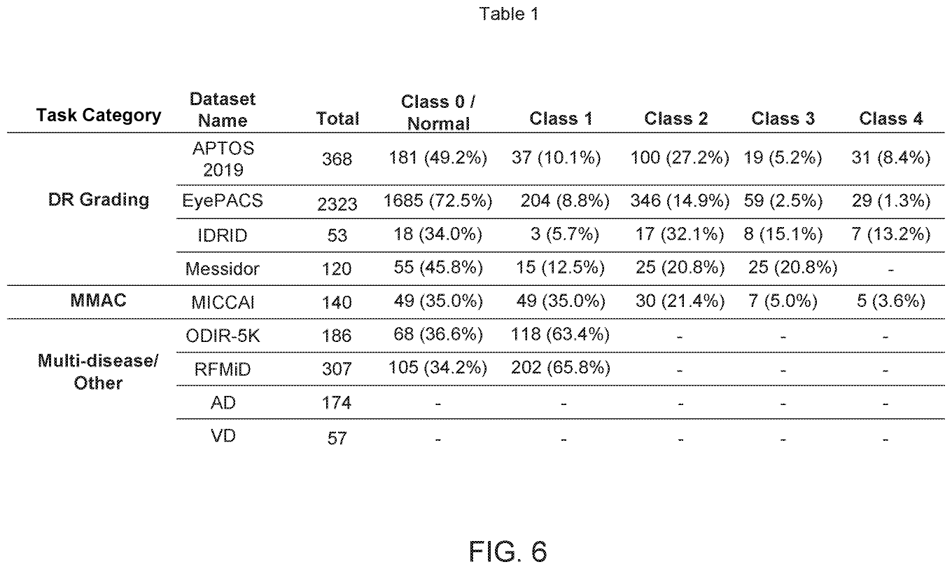

To ensure a balanced and comprehensive evaluation, a stratified sampling strategy was employed based on disease labels for data partitioning. The training and testing sets were derived from the same underlying data sources.
Stratified Selection: For each dataset, the testing set was selected according to the proportional distribution of diagnostic labels. This approach ensures that the class distribution in the testing set strictly mirrors that of the original dataset.
Comprehensive Label Coverage: By strictly adhering to label proportions during selection, the testing set was guaranteed to cover all disease categories and severity levels present in the training data.
Data Partitioning: The processed cohort was partitioned into training and evaluation subsets following a task-oriented strategy. To ensure robust performance assessment, 3,754 images were reserved for the independent evaluation set, with their specific distribution for disease diagnosis and lesion localization detailed in Table 1 presented in FIG. 6. Table 1 provides a summary of datasets and class distributions utilized in the evaluation. The datasets are categorized by diagnostic tasks (DR Grading, MMAC, and Binary Classification). Note that “−” denotes inapplicable categories. The remaining 38,061 (named 38K using above sections) high-quality images were utilized as the primary training cohort. This substantial scale of approximately 38K images provides a comprehensive foundation for model training, ensuring the integrity of cross-task feature learning without data leakage.

### 4.3 Implementation Details

The model was implemented using the PyTorch framework. The training process consisted of two distinct stages:
Stage 1 (Feature Alignment): the projector was trained for 1 epoch using a learning rate of 1e−3 and a batch size of 256. This stage utilized the mixup dataset comprising Retinal Concepts Alignment data and PMC-15M to align visual features with the LLM embedding space.
Stage 2 (Visual Instruction Tuning): both the projector and the LLM were fine tuned for 3 epochs. The learning rate was set to 2e−5 with a batch size of 128. This stage employed the mixup dataset of Retinal Concepts Tuning data and LLaVA-Med generic QA pairs.

All experiments were conducted on 8 NVIDIA A100 GPUs. The AdamW optimizer was used with a cosine annealing scheduler. The input image resolution was fixed at 336×336 pixels to match the vision encoder's pre-training settings.

### 4.4 Retinal Disease Diagnosis

The data used for retinal disease diagnosis exhibit heterogeneous disease distributions across different cohorts and tasks. To comprehensively evaluate the diagnostic capability of the proposed model, a two-stage evaluation protocol was designed consisting of a hard evaluation and a soft evaluation, targeting both deterministic diagnostic accuracy and the intrinsic characteristics of vision-language models (VLMs).

### 4.4.1 Hard Evaluation

The hard evaluation includes diagnostic tasks with explicit decision rules. Public retinal datasets were organized based on disease category and evaluation target.

For diabetic retinopathy (DR), four datasets—EyePACS, APTOS, Messidor, and IDRID—were combined and evaluated using a binary DR/Non-DR setting. Images annotated as DR grade 0 were counted as incorrect only when the model explicitly predicted DR. Predictions of other retinal diseases were not treated as DR errors, as the presence of non-DR pathologies could not be excluded from these datasets. The MMAC dataset, which targets myopia-related macular disease, was evaluated separately due to its different disease definition. Multi-disease datasets RFMiD and OIA-ODIR were used to evaluate performance under multiple co-existing pathologies. In addition, a private dataset, containing Alzheimer's disease-related (AD) and vascular dementia-related (VD) retinal images, was evaluated using a normal/disease classification setting.

### 4.4.2 Soft Evaluation

The disclosed embodiments provide a unified quantitative framework to evaluate model performance on a 100-point scale. The scoring protocol integrates diagnostic precision with linguistic quality, enforced by a strict safety penalty mechanism.
1. Diagnostic Accuracy (Primary Component) This module evaluates the clinical correctness of the diagnosis (20 points for each item):
   a. Anomaly Detection: A binary assessment of whether the model correctly distinguishes between pathological and healthy cases.
   b. Specific Disease Matching: A strict indicator verifying if the predicted diagnostic entity explicitly matches the ground truth (e.g., identifying “Diabetic Retinopathy” specifically, rather than generic “retinal abnormality”).
2. Linguistic and Clinical Quality: four qualitative metrics are adopted to assess the reliability of the generated report (15 points for each item):
   a. Professionalism: Use of precise medical terminology and maintenance of an objective clinical tone.
   b. Fluency: Grammatical correctness and syntactic flow.
   c. Directness: Absence of unnecessary preamble or evasive language.
   d. Conciseness: Information density and avoidance of redundancy.
3. Safety Penalty Mechanism (Hallucination) To penalize clinically dangerous fabrications, a Hallucination Penalty (20 points) was introduced. If the model generates content factually unrelated to the image (e.g., anatomical non-existence or fabricated objects), a 20-point deduction is applied to the final score.

### 4.4.3 Performance

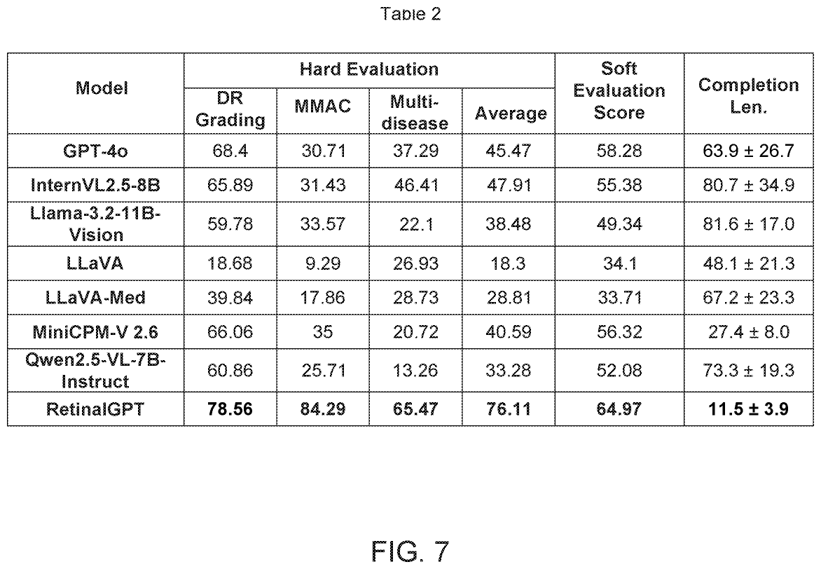

As detailed in Table 2 presented in FIG. 7, the proposed method demonstrates superior performance across both diagnostic precision and response quality benchmarks. Table 2 provides a quantitative comparison of diagnostic performance and response quality. “Hard Evaluation” reports the binary classification accuracy (%) for Diabetic Retinopathy (DR vs. No DR), Myopic Maculopathy (MMAC vs. Non-MMAC), and Multi-disease detection tasks (normal vs abnormal). The “Average” column represents the mean accuracy across these three hard tasks. “Soft Evaluation” denotes the cumulative score of the three qualitative metrics (Diagnostic Accuracy, Linguistic and Clinical Quality and Hallucination). “Completion Len.” indicates the average length of the generated responses (mean±standard deviation). In terms of hard evaluation, the disclosed embodiments achieve a commanding average accuracy of 76.11%, significantly outperforming the proprietary GPT-4o (45.47%) and the strongest open-source baseline, InternVL2.5-8B (47.91%). This advantage is particularly pronounced in the fine-grained MMAC classification task, where the disclosed embodiments attain 84.29% accuracy compared to GPT-4o's 30.71%, indicating that general-domain pre-training is insufficient for capturing subtle ophthalmic lesions. Beyond binary accuracy, the soft evaluation results confirm that the disclosed embodiments generate the most clinically reliable reports (Score: 64.97). Notably, this high performance is achieved with remarkable efficiency; the average response length of 11.5±3.9 words highlights the model's ability to provide direct, decisive diagnoses. In contrast, generalist MLLMs exhibit excessive verbosity (e.g., GPT-4o: ~64 words; InternVL: ~81 words), often relying on hedging or redundant descriptions rather than delivering the immediate clinical judgments required in real-world medical workflows.

### 4.5 Lesion Localization

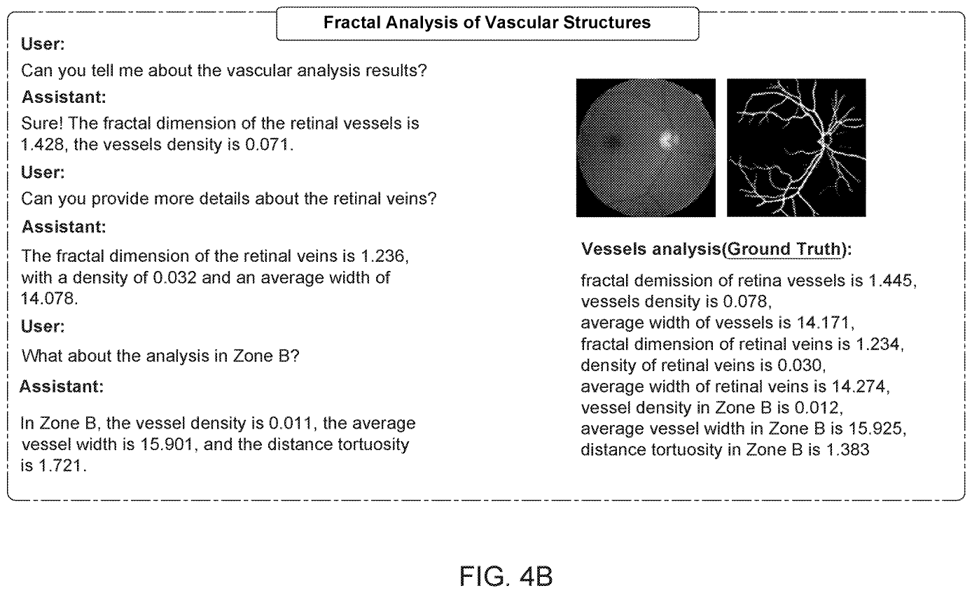

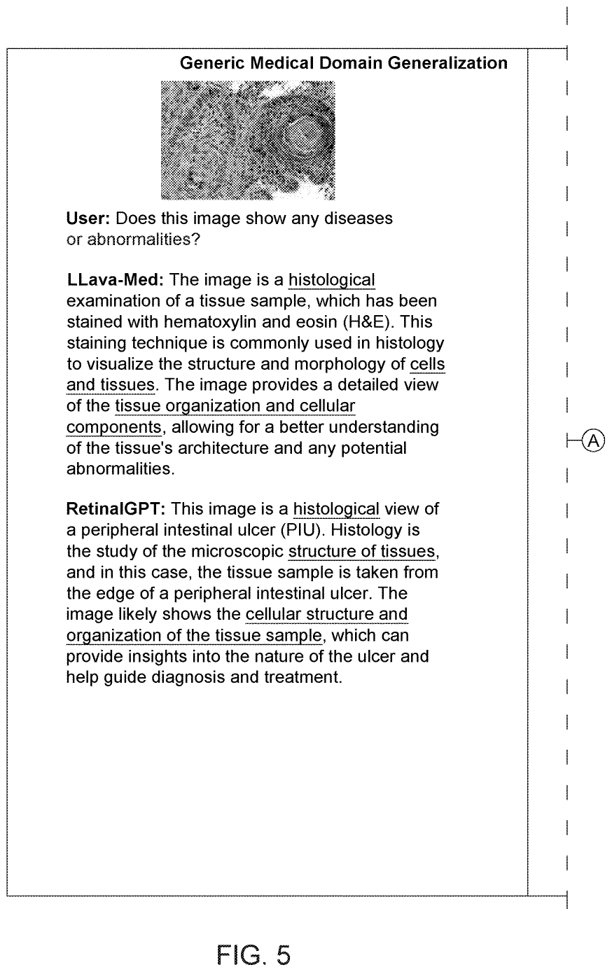

Beyond binary diagnosis, the model's grounding capabilities were evaluated. The process, shown in FIGS. 4A and 4B, uses the IDRiD and MICCAI datasets, which provide pixel-level annotations. Hit Rate, Precision, and Mean Match Distance were measured at two center-distance thresholds (α=0.5 and α=0.8).

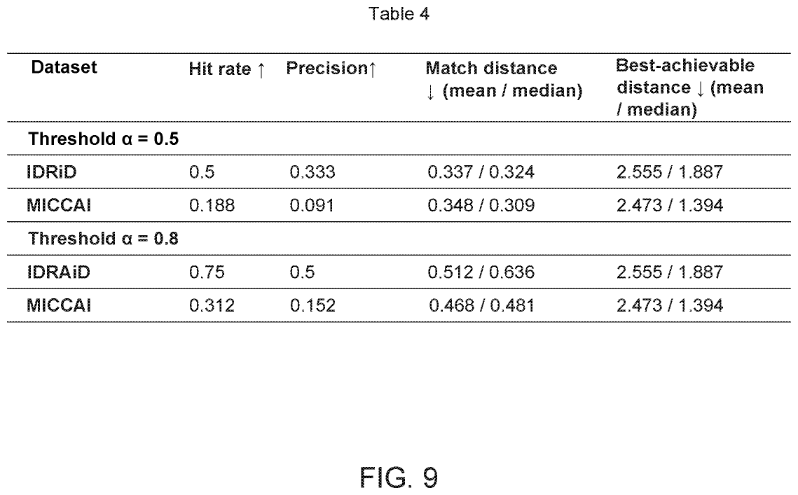

The quantitative results in Table 4 presented in FIG. 9 demonstrate that the disclosed embodiments can effectively localize lesions. Table 4 presents a quantitative evaluation of lesion localization performance on IDRiD and MICCAI datasets under two center-distance thresholds (α=0.5 and α=0.8). On IDRiD, the model achieved a Hit Rate of 0.750(α=0.8), significantly outperforming the results on MICCAI. This discrepancy is attributed to the nature of lesions; IDRiD contains more distinct lesions (e.g., hemorrhages, exudates), whereas MICCAI involves more subtle pathologies that are challenging even for human experts.

### 4.6 Quantitative Analysis of Vascular Structures

Beyond standard visual interpretation, the disclosed embodiments are designed to perform precise vascular structure analysis, retrieving key morphological feature values upon general query and providing detailed, region-specific metrics when further prompted. Qualitative Validation: As illustrated in FIG. 4B, the model's generated predictions were compared with ground-truth reference values derived from the automated AutoMorph pipeline. Visual inspection reveals a close alignment between textual outputs of the disclosed embodiments and the established reference standards, suggesting that the disclosed embodiments can provide accurate estimates of complex vascular features.
Quantitative Validation: To rigorously quantify this consistency, a systematic evaluation was conducted on a test set comprising 3,754 retinal fundus images (see Section 4.1). A comprehensive suite of metrics was reported to assess both error magnitude and statistical agreement:
Error-based Metrics: Mean Absolute Error (MAE), Median Absolute Error (MedAE), Root Mean Squared Error (RMSE) and Mean Absolute Percentage Error (MAPE) was used to capture the absolute and relative deviations between the model's predictions and the reference values.
Reliability Assessment: To evaluate the strength of the linear association and absolute agreement, Pearson's correlation coefficient (r) and the Intraclass Correlation Coefficient (ICC (2,1)) were employed, respectively.

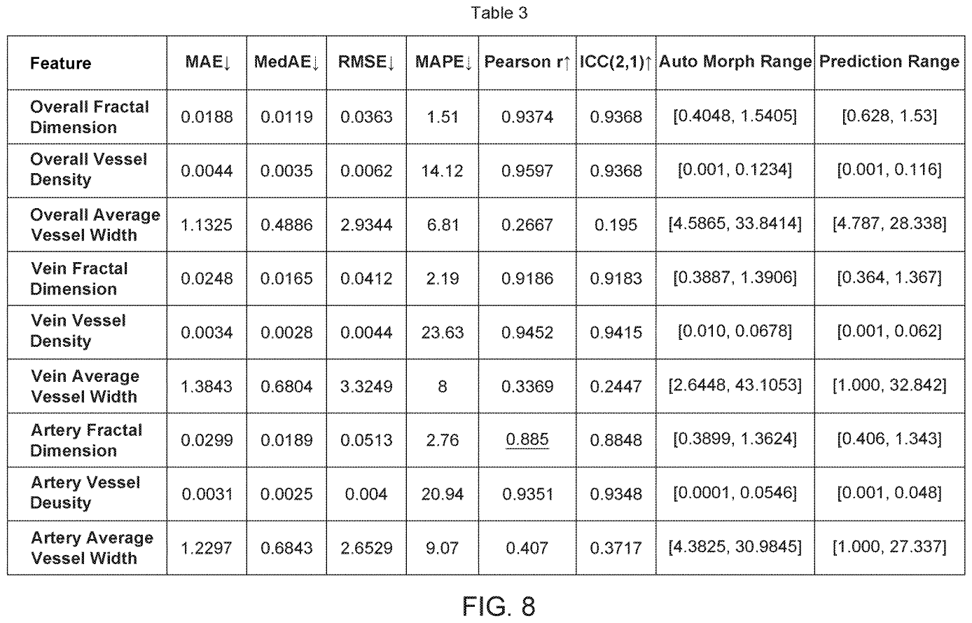

Together, these metrics offer a holistic view of the model's accuracy and consistency. The detailed benchmarking results, summarized in Table 3 presented in FIG. 8, establish a systematic baseline for vascular biomarker estimation using multimodal large language models. Table 3 provides a quantitative comparison between predicted vascular features and AutoMorph reference measurements. The table reports both error-based metrics (MAE, MedAE, RMSE, MAPE) and correlation-based metrics (Pearson r, ICC (2,1)) across nine representative vascular biomarkers, including overall, venous, and arterial features. Lower error values and higher correlation coefficients indicate better agreement with the reference, demonstrating the model's capability for accurate vascular feature estimation in retinal images.

### 4.7 Generalization to Generic Biomedical Domains

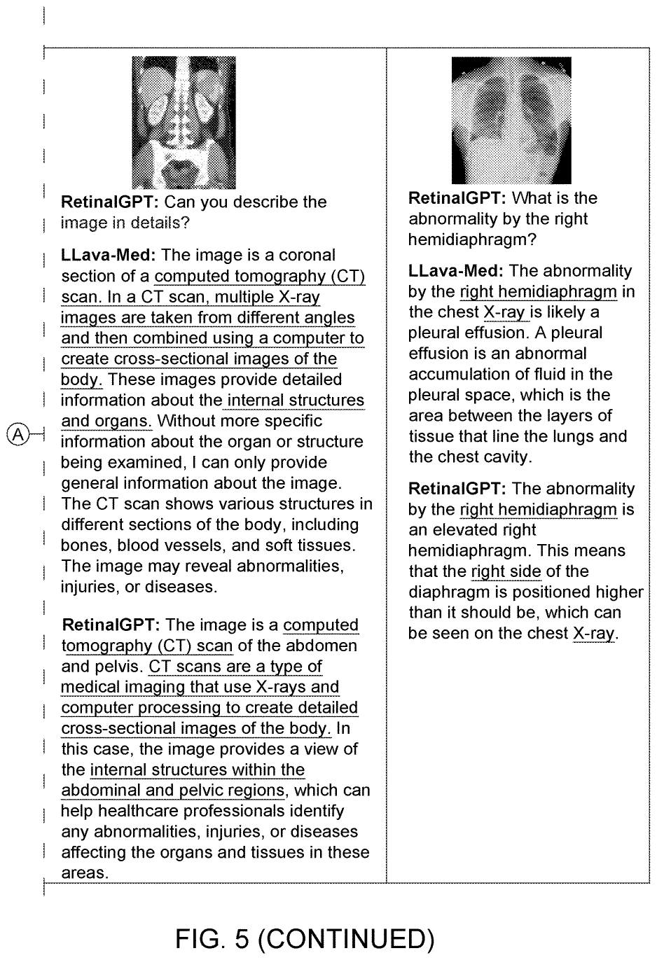

A common challenge in developing specialized medical AI assistants is “catastrophic forgetting,” where fine-tuning on a specific domain (e.g., ophthalmology) leads to a degradation of general medical knowledge. To evaluate whether the proposed Mixup Instruction-Tuning strategy effectively mitigates this issue, the model's performance on generic biomedical tasks beyond retinal imaging was assessed. A comparative qualitative analysis was conducted against LLaVA-Med13 on diverse medical imaging modalities, including Chest X-rays (CXR), CT scans, and histological slides. As illustrated in FIG. 5, the model demonstrates robust generalization capabilities.
Case Study (Chest X-ray): When presented with a chest X-ray exhibiting an abnormality near the right hemidiaphragm, the model accurately identified it as an “elevated right hemidiaphragm” and provided a clinically coherent explanation (“the right side of the diaphragm is positioned higher than it should be”).
Comparison: This response is highly consistent with LLaVA-Med's diagnosis (identifying related pleural abnormalities), confirming that the model retains the foundational medical visual understanding acquired during the alignment stage.

These results indicate that the two-stage training strategy—specifically the incorporation of the 60K Generic QA dataset during instruction tuning—successfully balances domain specialization with general medical versatility. The disclosed embodiments serve as a dual-purpose tool: a specialist in retinal quantitative analysis and a competent generalist in broader biomedical contexts.

### 5. Discussion

### 5.1 Performance Comparison and Domain Alignment

The experimental results across eight benchmark datasets indicate that the model achieves higher diagnostic accuracy compared to general-domain MLLMs (e.g., GPT-4o) and medical-domain adaptations (e.g., LLaVA-Med). General MLLMs, while possessing strong logical reasoning, often lack the specific visual alignment required for retinal pathologies, resulting in lower performance on fine-grained tasks like DR grading. Similarly, existing medical VLMs, which are primarily trained on broad biomedical literature, struggle with the specific morphological details of the retina. The disclosed embodiments address this by explicitly aligning visual features with clinical concepts through the constructed instruction-tuning datasets (RCA and RCT), thereby enhancing performance in specialized ophthalmic tasks.

### 5.2 Interpretability Via Quantitative Analysis

A distinction of the framework is the integration of quantitative vascular analysis into the diagnostic process. Unlike “black-box” models that output only classification labels, the model generates interpretable metrics such as vessel density and fractal dimension. The high correlation (r>0.93) observed between the model's outputs and standard automated measurements (AutoMorph) suggests that the model effectively internalizes these morphological features during training. This capability is relevant for clinical applications where quantitative tracking of disease progression is required, such as in hypertensive retinopathy.

### 5.3 Generalization and Catastrophic Forgetting

The qualitative evaluation on non-retinal images (e.g., Chest X-rays) demonstrates that the model retains general biomedical knowledge. This suggests that the two-stage mixup training strategy effectively mitigates catastrophic forgetting, a common issue when fine-tuning on specialized datasets. The model maintains its utility as a general medical assistant while offering specialized capabilities in ophthalmology.

### 5.4 Limitations and Future Work

The above analysis is currently restricted to 2D Color Fundus Photography. Integrating other modalities, such as 3D Optical Coherence Tomography (OCT), would provide a more comprehensive assessment. Additionally, it was observed that in the initial turn of dialogue, the model's responses can be formulaic regarding modality identification, likely due to limited diversity in the initial QA prompt templates. Future work will aim to expand the instruction diversity and incorporate multi-modal data to further enhance the model's clinical applicability.

Thus, the disclosed embodiments provide for a method and system for constructing a clinical dataset for retinal image analysis, comprising performing an instruction-tuning technique using a two-stage approach for adapting general-domain vision-language models (VLMs) for retinal domain tasks. The two-stage approach involves broadening a vocabulary of aligned image-text tokens to incorporate both retinal-specific knowledge and generic biomedical information to preserve broader knowledge, and performing mixup instruction-tuning on domain-specific knowledge and generic knowledge to retain the broader knowledge.

According to the disclosed embodiments, broadening a vocabulary of aligned image-text tokens to incorporate both retinal-specific knowledge and generic biomedical information to preserve broader knowledge, comprises aligning image features of retinal images and other biomedical visual concepts with corresponding textual word embeddings from a pre-trained language model, thereby expanding the vocabulary of aligned image-text tokens to include both retinal-specific knowledge and broader biomedical information.

According to the disclosed embodiments, performing mixup instruction-tuning on domain-specific knowledge and generic knowledge to retain the broader knowledge, comprises mixup instruction-tuning indicating that a training process integrates both generic medical conceptions understanding data and task-specific instruction tuning retinal data.

The disclosed embodiments further provide for a method and system for receiving a plurality of retinal images, extracting clinical feature descriptions, including disease labels, lesion bounding boxes, and fractal features, from the plurality of retinal images, fractal analyzing each retinal image to extract features including fractal dimension of vessels, vessel density, branch angle, curvature tortuosity, and morphological characteristics relevant to vascular structure, and statistically analyzing the extracted features to examine their relationship with disease labels and to ensure the extracted features are clinically relevant.

According to the disclosed embodiments, extracting clinical feature descriptions, including disease labels, comprises collecting a disease label for each retinal image based on medical annotations.

According to the disclosed embodiments, fractal analyzing each retinal image to extract morphological characteristics relevant to vascular structure, comprises fractal analyzing each retinal image using automated retinal vascular morphology quantification via a deep learning pipeline (AutoMorph) and a dataset and benchmark for retinal vessels branching angle detection (RBAD).

The disclosed embodiments further comprise receiving ophthalmologist input to confirm the importance of each extracted feature and ophthalmologist input to retain extracted features that contribute to disease characterization remove redundant or less relevant extracted features.

The disclosed embodiments further comprise evaluating image quality X_{q} of each retinal image and assigning one of three labels: good, usable, and reject, to each retinal images based on the image quality.

According to the disclosed embodiments, extracting lesion bounding boxes comprises locating lesions and generating the bounding boxes in a format (xₘᵢₙ, yₘᵢₙ, xₘₐₓ, yₘₐₓ).

The disclosed embodiments further comprise integrating extracted disease labels X_{d}, quality assessments X_{q}, vascular features X_{f}, lesion information Xₗ and a modality of each retinal image Xₘ(Color Fundus Photograph) into a structured description for the retinal image, as (X_{f}, X_{q}, X_{d}, Xₘ, Xₗ). For each retinal image, the disclosed embodiments further comprise a single-round instruction-following dialogue about the retinal image using the disease label X_{d} and the image modality Xₘ, without utilizing the retinal image, by leveraging GPT-4.

The disclosed embodiments further comprise constructing a sample question X_{q} to inquire about information about a retinal image, and obtaining a corresponding answer Xₘ, X_{d} comprising: Human: X_{q}<STOP>, Assistant: XₘX_{d}<STOP>. The disclosed embodiments further comprise generating instruction data (X_{f}, X_{q}, X_{d}, Xₘ, Xₗ) from the extracted features.

### 6. Conclusion

The disclosed embodiments, referred to herein as RetinalGPT, provide a multimodal conversational assistant tailored for quantitative analysis of retinal fundus images. By constructing a specialized instruction-following dataset grounded in clinically relevant morphological features and adopting a two-stage instruction tuning strategy, the disclosed embodiments address key limitations of existing multimodal language models in ophthalmic image analysis. Extensive evaluations across eight benchmark datasets demonstrate that RetinalGPT achieves consistently improved performance in retinal disease assessment, lesion localization, and quantitative vascular characterization compared with general-domain baselines. More importantly, the results indicate that explicitly incorporating quantitative clinical priors into multimodal training facilitates interpretable image-centered analysis, enabling systematic exploration of both localized pathology and global vascular patterns. Together, these findings highlight the potential of quantitatively grounded multimodal assistants to support retinal image analysis and translational ophthalmic research, offering a reproducible pathway toward clinically meaningful and interpretable medical AI systems.

What is claimed is:

1. A method for constructing a clinical dataset for retinal image analysis, comprising:
performing an instruction-tuning technique using a two-stage approach for adapting general-domain vision-language models (VLMs) for retinal domain tasks, comprising:
broadening a vocabulary of aligned image-text tokens to incorporate both retinal-specific knowledge and generic biomedical information to preserve broader knowledge; and
performing mixup instruction-tuning on domain-specific knowledge and generic knowledge to retain the broader knowledge.

2. The method of claim 1, wherein broadening a vocabulary of aligned image-text tokens to incorporate both retinal-specific knowledge and generic biomedical information to preserve broader knowledge, comprises aligning image features of retinal images and other biomedical visual concepts with corresponding textual word embeddings from a pre-trained language model, thereby expanding the vocabulary of aligned image-text tokens to include both retinal-specific knowledge and broader biomedical information.

3. The method of claim 1, wherein performing mixup instruction-tuning on domain-specific knowledge and generic knowledge to retain the broader knowledge, comprises mixup instruction-tuning indicating that a training process integrates both generic medical conceptions understanding data and task-specific instruction tuning retinal data.

4. A method, comprising:
receiving a plurality of retinal images;
extracting clinical feature descriptions, including disease labels, lesion bounding boxes, and fractal features, from the plurality of retinal images;
fractal analyzing each retinal image to extract features including fractal dimension of vessels, vessel density, branch angle, curvature tortuosity, and morphological characteristics relevant to vascular structure; and
statistically analyzing the extracted features to examine their relationship with disease labels and to ensure the extracted features are clinically relevant.

5. The method of claim 4, wherein extracting clinical feature descriptions, including disease labels comprises collecting a disease label for each retinal image based on medical annotations.

6. The method of claim 4, wherein fractal analyzing each retinal image to extract morphological characteristics relevant to vascular structure, comprises fractal analyzing each retinal image using automated retinal vascular morphology quantification via a deep learning pipeline (AutoMorph) and a dataset and benchmark for retinal vessels branching angle detection (RBAD).

7. The method of claim 4, further comprising receiving ophthalmologist input to confirm the importance of each extracted feature.

8. The method of claim 6, further comprising ophthalmologist input to retain extracted features that contribute to disease characterization remove redundant or less relevant extracted features.

9. The method of claim 4, further comprising:
evaluating image quality X_{q} of each retinal image; and,
assigning one of three labels: good, usable, and reject, to each retinal images based on the image quality.

10. The method of claim 4, wherein extracting lesion bounding boxes comprises locating lesions and generating the bounding boxes in a format (xₘᵢₙ, yₘᵢₙ, xₘₐₓ, yₘₐₓ).

11. The method of claim 4, further comprises integrating extracted disease labels X_{d}, quality assessments X_{q}, vascular features X_{f}, lesion information Xₗ and a modality of each retinal image Xₘ(Color Fundus Photograph) into a structured description for the retinal image, as (X_{f}, X_{q}, X_{d}, Xₘ, Xₗ).

12. The method of claim 11, further comprising: for each retinal image, generating a single-round instruction-following dialogue about the retinal image using the disease label X_{d} and the image modality Xₘ, without utilizing the retinal image, by leveraging GPT-4.

13. The method of claim 11, further comprising:
constructing a sample question X_{q} to inquire about information about a retinal image, and obtaining a corresponding answer Xₘ, X_{d} comprising:
Human: X_{q}<STOP>
Assistant: XₘX_{d}<STOP>.

14. The method of claim 12, further comprising generating instruction data (X_{f}, X_{q}, X_{d}, Xₘ, Xₗ) from the extracted features.

15. A system comprising:
a memory to store instructions;
a processor to execute the instructions stored in the memory to perform the following operations:
receiving a plurality of retinal images;
extracting clinical feature descriptions, including disease labels, lesion bounding boxes, and fractal features, from the plurality of retinal images;
fractal analyzing each retinal image to extract features including fractal dimension of vessels, vessel density, branch angle, curvature tortuosity, and morphological characteristics relevant to vascular structure; and
statistically analyzing the extracted features to examine their relationship with disease labels and to ensure the extracted features are clinically relevant.

16. The system of claim 15, wherein extracting clinical feature descriptions, including disease labels comprises collecting a disease label for each retinal image based on medical annotations.

17. The system of claim 15, wherein fractal analyzing each retinal image to extract morphological characteristics relevant to vascular structure, comprises fractal analyzing each retinal image using automated retinal vascular morphology quantification via a deep learning pipeline (AutoMorph) and a dataset and benchmark for retinal vessels branching angle detection (RBAD).

18. A non-transitory computer readable storage media having instructions stored thereupon that, when executed by a system having at least a processor and a memory therein, cause the processor to perform the following operations:
receiving a plurality of retinal images;
extracting clinical feature descriptions, including disease labels, lesion bounding boxes, and fractal features, from the plurality of retinal images;
fractal analyzing each retinal image to extract features including fractal dimension of vessels, vessel density, branch angle, curvature tortuosity, and morphological characteristics relevant to vascular structure; and
statistically analyzing the extracted features to examine their relationship with disease labels and to ensure the extracted features are clinically relevant.

19. The non-transitory computer readable storage media of claim 18, wherein extracting clinical feature descriptions, including disease labels comprises collecting a disease label for each retinal image based on medical annotations.

20. The non-transitory computer readable storage media of claim 18, wherein fractal analyzing each retinal image to extract morphological characteristics relevant to vascular structure, comprises fractal analyzing each retinal image using automated retinal vascular morphology quantification via a deep learning pipeline (AutoMorph) and a dataset and benchmark for retinal vessels branching angle detection (RBAD).
